(19) United States
(12) Patent Application Publication
(10) Pub. No.: US 2007/0048371 A1
(43) Pub. Date: Mar. 1, 2007

(54) Pharmaceutical formulations/composition of guanfacine suitable for single dose form administration daily

(76) Inventors: Amir H. Shojaei, Phoenixville, PA (US); Michael Pennick, Oxford (GB)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/493,914

(22) Filed: Jul. 27, 2006

Related U.S. Application Data
(60) Provisional application No. 60/702,982, filed on Jul. 28, 2005.

(51) Int. Cl. A61K 9/20; A61K 31/165

(52) U.S. Cl. 424/464; 514/613

(57) ABSTRACT

Therapeutic formulations of guanfacine in a once a day single dose form which have useful overall tablet sizes are disclosed.

### RELATED APPLICATIONS

This application claims the benefit of the filing date of U.S. Provisional Application Ser. No. 60/702,982 filed Jul. 28, 2005.

This application is related to concurrently filed application, identified by Attorney Docket No. SHIRE-0519-V01, and entitled “Modified Release Tablet Formulations with Enhanced Mechanical Properties.” The entire contents of the foregoing patent application is expressly incorporated by reference in its entirety.

### BACKGROUND OF THE INVENTION

Guanfacine is useful for treating ADHD as an alternative to stimulant medications, mild to moderate high blood pressure (hypertension), heroin withdrawal, certain problems in difficult pregnancies, sleep disorders, among others. Other indications are being tested. This drug is especially useful in children who have both ADHD and conduct disorder. See U.S. Pat. No. 6,811,794 and U.S. Pat. No. 6,287,599 which disclose formulations of guanfacine, whose disclosures are incorporated by reference herein in their entirety.

Guanfacine hydrochloride is a centrally acting antihypertensive with α₂-adrenoceptor agonist properties, sold in tablet form for oral administration under the brand name Tenex®. The chemical name of guanfacine hydrochloride is N-amidino-2-(2,6-dichlorophenyl) acetamide hydrochloride and its has the molecular formula, C₉H₁₀Cl₃N₃O. It is in the form of a white to off-white powder that is sparingly soluble in water and alcohol and slightly soluble in acetone. Each commercial tablet for oral administration contains guanfacine hydrochloride equivalent to 1 mg or 2 mg guanfacine. The tablets additionally contain anhydrous lactose, microcrystalline cellulose, povidone, stearic acid and colorants.

Guanfacine may cause the following side effects: dry mouth, constipation, drowsiness, dizziness, headache, difficulty sleeping (insomnia), dry eyes, nausea, vomiting, skin rash or itching, fatigue, indigestion, diarrhea, slow heartbeat, decreased sexual ability, unusual tiredness or weakness, confusion, mental depression, amnesia, liver toxicity, leg cramp, sudden high blood pressure (if stopped abruptly).

For purposes of simplicity, the term “guanfacine” is understood to refer to both the compound guanfacine and any of its salts including guanfacine hydrochloride unless specifically stated otherwise or listed separately from each other.

### THE INVENTION

This invention provides methods, compositions, and formulations that are useful in the treatment of any of the indications for guanfacine. The methods of the invention include administering to a subject a once a day, oral therapeutic composition or formulation containing guanfacine in the prescribed dose, e.g., 1 mg, 1.5 mg, 2 mg, 2.5 mg, 3 mg, 3.5 mg, etc., in a single dose form, e.g., a single tablet, which is effective in a once a day regimen and also has a size, e.g., tablet weight, small enough to be acceptable for oral administration, e.g., allowing for easy swallowing by a patient. Accordingly, the compositions and methods of the invention are useful for treating, controlling or affecting ADHD, hypertension, heroin withdrawal, certain problems in difficult pregnancies, sleep disorders, etc. The formulations also have good manufacturability, scalability and robustness.

An aspect of the invention is a pharmaceutical composition comprising guanfacine and a pharmaceutically acceptable vehicle in a single, once a day discrete dose form for oral administration which is effective in a once a day regimen and also has a size acceptable for patients. For example, typical acceptable sizes, in terms of tablet weight, are for a 1 mg dose, up to 170 mg, for a 2 mg dose, up to 340 mg, for a 2.5 mg dose, up to 255 mg, for a 3 mg dose, up to 225 mg, for a 3.5 mg dose, up to 245 mg, and for a 4 mg dose, up to 300 mg. Sizes may vary with the geometric shape that is acceptable for oral administration, for example.

Round tablets often have diameters up to 0.4 inch, preferably up to 0.35 inch, e.g., 0.1500 inch to 0.3125 inch, preferably 0.2000 to 0.3125 inch, more preferably 0.2813 to 0.3125 inch with a thickness of up to 0.2 inch, preferably up to 0.15 inch, e.g., 0.1000 to 0.1400 inch. If the tablet is oval it often has dimensions of up to 0.6 inch (length) by up to 0.2 inch (width), preferably up to 0.55 inch (length) by up to 0.15 inch (width), e.g., 0.5400 (length) by 0.1200 (width) inch, preferably 0.5000 (length) by 0.1800 (width), more preferably 0.4860 (length) by 0.2400 (width) inch with a thickness of up to 0.25 inch, preferably up to 0.22 inch, e.g., 0.140 to 0.20 inch. The shape of the discrete oral dose form can be oval or round or other suitable shape. When the oral dose form, for example, a tablet, is round, the characterizing dimension usually is its diameter and thickness, and when it is oval, the characterizing dimension is usually its length, width, and thickness.

Another aspect of the invention is such a pharmaceutical composition that has a total weight of, for example, up to about 340 mg, e.g., 100 to 300 mg, preferably 130 to 300 mg, more preferably 150 to 300 mg.

In a further aspect the preferred tablet of the invention has a hardness of 4.0 to 11.0 kp, preferably 4.5 to 9.5 kp, more preferably 4.5 to 8.0 kp, measured using a Schleuniger tablet hardness tester.

Another aspect is a method for the treatment of ADHD with the pharmaceutical composition described above, for example, in adults (18+ years), and children, e.g., about ages 6-12 and about ages 13-17. Thus, this invention includes tablets that are able to achieve an effective pk profile, and size, e.g., weight. In another aspect, the tablets also achieve sufficient compressibility to attain desired tablet hardness values.

Advantages of being able to administer guanfacine in a single once a day dose form include lessening the chance for a patient missing a prescribed administration regimen, e.g., no chance of forgetting taking a pill twice, three times, etc., daily; ease of administration to a child daily versus more than once if the child resists or generally objects to taking medication each time administered; less interference with daily activities, e.g., school, sports practice etc. for kids, work, etc.; and convenience; etc.

Another aspect of the invention is the pharmaceutical formulation of the invention having a mean plasma concentration pharmacokinetic (PK) profile such that, when the formulation is administered in a dose of 1 mg of guanfacine to fasted healthy adult subjects, said PK profile has at least one of the following parameters: an AUC₀₋ₗₐₛₜ of about 29.3±8.8 ng·h/mL, an AUC_{∞} of about 32.4±8.8 ng·h/mL, a mean Cₘₐₓ of about 0.98±0.26 ng/mL, and a median Tₘₐₓ of about 6 hours (each parameter being within ±50%, more preferably ±40% and particularly preferably ±20% of said respective value (irrespective of stated±value), and when the formulation is administered in a dose amount of 2 mg or another dose amount whose PK profile is linearly correlated with that of a 1 mg dose, an AUC₀₋ₗₐₛₜ, AUC_{∞}, and Cₘₐₓ linearly related to those for the 1 mg dose. The t_{1/2}, measured for the 1 mg formulation, is 17.5±3.8 hours.

Another aspect of the invention is the pharmaceutical formulation of the invention having a mean plasma concentration pharmacokinetic (PK) profile such that, when the formulation is administered in a dose of 2.5 mg of guanfacine to fasted healthy adult subjects, said PK profile has at least one of the following parameters: an AUC₀₋ₗₐₛₜ of about 81.3±35.4 ng·h/mL, an AUC_{∞} of about 85.0±37.4 ng·h/mL, a mean Cₘₐₓ of about 2.49±0.93 ng/mL, and a median Tₘₐₓ of about 6 hours (each parameter being within ±50%, more preferably ±40% and particularly preferably ±20% of said respective value (irrespective of stated±value), and when the formulation is administered in a dose amount whose PK profile is linearly correlated with that of a 2.5 mg dose, an AUC₀₋ₗₐₛₜ, AUC_{∞}, and Cₘₐₓ linearly related to those for the 2.5 mg dose. The t_{1/2}, measured for the 1 mg formulation, is 16.7±7.4 hours.

Another aspect of the invention is the pharmaceutical formulation of the invention having a mean plasma concentration pharmacokinetic (PK) profile such that, when the formulation is administered in a dose of 4 mg of guanfacine to fasted healthy adult subjects, said PK profile has at least one of the following parameters: an AUC₀₋ₗₐₛₜ of about 120±41.5 ng·h/mL, an AUC_{∞} of about 125±46.0 ng·h/mL, a mean Cₘₐₓ of about 3.58±1.39 ng/mL, and a median Tₘₐₓ of about 5 hours (each parameter being within ±50%, more preferably ±40% and particularly preferably ±20% of said respective value (irrespective of stated±value), and when the formulation is administered in a dose amount of 3 mg, 3.5 mg or another dose amount whose PK profile is linearly correlated with that of a 4 mg dose, an AUC₀₋ₗₐₛₜ, AUC_{∞}, and Cₘₐₓ linearly related to those for the 4 mg dose. The t_{1/2}, measured for the 1 mg formulation, is 17.1±5.5 hours.

Another aspect of the invention is the pharmaceutical formulation of the invention having a mean plasma concentration pharmacokinetic (PK) profile such that, when the formulation is administered in a dose of 2 mg of guanfacine to fasted patients ages 6-12 suffering from ADHD, said PK profile has at least one of the following parameters: an AUC₀₋ₗₐₛₜ of about 56.88±22.05 ng·h/mL, an AUC_{∞} of about 65.20±23.88 ng·h/mL, a mean Cₘₐₓ of about 2.55±1.03 ng/mL, and a median Tₘₐₓ of about 5 hours (each parameter being within ±50%, more preferably ±40% and particularly preferably ±20% of said respective value (irrespective of stated±value), and when the formulation is administered in a dose amount of 1 mg or another dose amount whose PK profile is linearly correlated with that of a 2 mg dose, an AUC₀₋ₗₐₛₜ, AUC_{∞}, and Cₘₐₓ linearly related to those for the 2 mg dose. The t_{1/2}, measured for the 2 mg formulation, is 14.4±2.4 hours.

Another aspect of the invention is the pharmaceutical formulation of the invention having a mean plasma concentration pharmacokinetic (PK) profile such that, when the formulation is administered in a dose of 2 mg of guanfacine to fasted patients ages 13-17 suffering from ADHD, said PK profile has at least one of the following parameters: an AUC₀₋ₗₐₛₜ of about 42.74±12.85 ng·h/mL, an AUC_{∞} of about 47.25±13.69 ng·h/mL, a mean Cₘₐₓ of about 1.69±0.43 ng/mL, and a median Tₘₐₓ of about 5 hours (each parameter being within ±50%, more preferably ±40% and particularly preferably +20% of said respective value (irrespective of stated±value), and when the formulation is administered in a dose amount of 1 mg or another dose amount whose PK profile is linearly correlated with that of a 2 mg dose, an AUC₀₋ₗₐₛₜ, AUC_{∞}, and Cₘₐₓ linearly related to those for the 2 mg dose. The t_{1/2}, measured for the 2 mg formulation, is 17.9±5.8 hours.

Formulations of the invention typically contain about 0.1 to about 5% (w/w) of guanfacine, preferably 0.25-5% (w/w), more preferably 0.3-4% (w/w), 0.33-3.5% (w/w), 0.5-3% (w/w), 0.75-2% (w/w), etc. Amounts of other ingredients will vary with the formulation technology used and the PK profiles to be achieved, as will the amount of guanfacine, all as shown herein.

All patients can get any of the 1, 2, 2.5, 3, 3.5 or 4 mg once a day formulations based on desired PK profiles and effectiveness to be achieved. All patients could get the 3 mg dose (if they are not adequately maintained on lower doses). The dose for more mature patients, e.g., teenagers and adults, and also for heavier children may be titrated to 3.5 or 4 mg, for example.

Preferred formulations that achieve the desired PK profile are provided in Table 1. More preferred formulations that achieve the desired PK profile are provided in Table 2.

TABLE 1

| | 1 mg Formulation | | 2 mg Formulation | | 2.5 mg Formulation | |
|---|---|---|---|---|---|---|
| Component | mg/tablet | % w/w | mg/tablet | % w/w | mg/tablet | % w/w |
| Guanfacine Hydrochloride, | 1.14 | 0.76-0.88 | 2.28 | 0.76-0.88 | 2.85 | 1.27-1.42 |
| USP/NF | | | | | | |
| Hypromellose 2208, USP/NF | 17.34-20.01 | 13.34 | 34.68-40.02 | 13.34 | 26.68-30.02 | 13.34 |
| (Methocel ® K4M CR Premium) | | | | | | |
| High Density Silicified | 20.00-26.00 | 15.38-17.33 | 40.00-51.99 | 15.38-17.33 | 34.19-38.99 | 17.10-17.33 |
| Microcrystalline Cellulose | | | | | | |
| (Prosolv HD90 ™) | | | | | | |
| Lactose Monohydrate, Povidone, | 22.02-25.37 | 16.91-16.94 | 44.04-50.73 | 16.91-16.94 | 31.98-35.78 | 15.90-15.99 |
| Crospovidone Granulated Blend | | | | | | |
| (Ludipress ®) | | | | | | |
| Methacrylic Acid Copolymer, | 45.00-50.00 | 33.33-34.61 | 90.00-99.99 | 33.33-34.61 | 66.66-74.99 | 33.33 |
| Type C, USP/NF (Eudragit ® L | | | | | | |
| 100-55) | | | | | | |
| Fumaric Acid, FCC | 6.50-7.50 | 5.00 | 13.00-15.00 | 5.00 | 10.00-11.25 | 5.00 |
| Atomized Glyceryl Behenate, | 18.00-20.00 | 13.33-13.85 | 36.00-39.99 | 13.33-13.85 | 26.66-29.99 | 13.33 |
| USP/NF (Compritol ® 888 ATO) | | | | | | |
| Pigment Blend Powder* | — | — | — | — | 1.00-1.13 | 0.50 |
| Total | 130.00-150.00 | 100.00 | 260.00-300.00 | 100.00 | 200.00-225.00 | 100.00 |
| Tablet Size/Shape | 9/32″ | Round | 0.486″ × 0.240″ | Oval | Round | 5/16″ |

| | 3 mg Formulation | | 3.5 mg Formulation | | 4 mg Formulation | |
|---|---|---|---|---|---|---|
| Component | mg/tablet | % w/w | mg/tablet | % w/w | mg/tablet | % w/w |
| Guanfacine Hydrochloride, | 3.42 | 1.71-1.90 | 3.99 | 1.71-1.90 | 4.56 | 1.71-1.90 |
| USP/NF | | | | | | |
| Hypromellose 2208, USP/NF | 18.00-20.00 | 10.00 | 21.00-23.33 | 10.00 | 24.00-26.67 | 10.00 |
| (Methocel ® K4M CR Premium) | | | | | | |
| High Density Silicified | 37.88-42.78 | 21.04-21.39 | 44.18-49.91 | 21.04-21.39 | 50.49-57.04 | 21.04-21.39 |
| Microcrystalline Cellulose | | | | | | |
| (Prosolv HD90 ™) | | | | | | |
| Lactose Monohydrate, Povidone, | 38.80-42.80 | 21.40-21.55 | 45.25-49.93 | 21.40-21.55 | 51.72-57.07 | 21.40-21.55 |
| Crospovidone Granulated Blend | | | | | | |
| (Ludipress ®) | | | | | | |
| Methacrylic Acid Copolymer, | 45.00-50.00 | 25.00 | 52.50-58.33 | 25.00 | 60.00-66.67 | 25.00 |
| Type C, USP/NF (Eudragit ® L | | | | | | |
| 100-55) | | | | | | |
| Fumaric Acid, FCC | 9.00-10.00 | 5.00 | 10.50-11.67 | 5.00 | 12.00-13.33 | 5.00 |
| Atomized Glyceryl Behenate, | 27.00-30.00 | 15.00 | 31.50-35.00 | 15.00 | 36.00-40.00 | 15.00 |
| USP/NF (Compritol ® 888 ATO) | | | | | | |
| Pigment Blend Powder* | 0.90-1.00 | 0.50 | 1.05-1.17 | 0.50 | 1.20-1.33 | 0.50 |
| Total | 180.00-200.00 | 100.00 | 210.00-233.33 | 100.00 | 240.00-266.66 | 100.00 |
| Tablet Size/Shape | Round | 5/16″ | Round | 5/16″ | 0.486″ × 0.240″ | Oval |

*2.5 mg: Blue pigment; 3 mg: Green pigment; 3.5 mg: Red pigment; 4 mg: Green pigment

TABLE 2

| | 1 mg Formulation | | 2 mg Formulation | | 2.5 mg Formulation | |
|---|---|---|---|---|---|---|
| Component | mg/tablet | % w/w | mg/tablet | % w/w | mg/tablet | % w/w |
| Guanfacine Hydrochloride, | 1.14 | 0.76 | 2.28 | 0.76 | 2.85 | 1.27 |
| USP/NF | | | | | | |
| Hypromellose 2208, USP/NF | 20.01 | 13.34 | 40.02 | 13.34 | 30.02 | 13.34 |
| (Methocel ® K4M CR Premium) | | | | | | |
| High Density Silicified | 26.00 | 17.33 | 51.99 | 17.33 | 38.99 | 17.33 |
| Microcrystalline Cellulose | | | | | | |
| (Prosolv HD90 ™) | | | | | | |
| Lactose Monohydrate, | 25.37 | 16.91 | 50.73 | 16.91 | 35.78 | 15.90 |
| Povidone, Crospovidone | | | | | | |
| Granulated Blend (Ludipress ®) | | | | | | |
| Methacrylic Acid Copolymer, | 50.00 | 33.33 | 99.99 | 33.33 | 74.99 | 33.33 |
| Type C, USP/NF (Eudragit ® L | | | | | | |
| 100-55) | | | | | | |
| Fumaric Acid, FCC | 7.50 | 5.00 | 15.00 | 5.00 | 11.25 | 5.00 |
| Atomized Glyceryl Behenate, | 20.00 | 13.33 | 39.99 | 13.33 | 29.99 | 13.33 |
| USP/NF (Compritol ® 888 ATO) | | | | | | |
| Pigment Blend Powder* | — | — | — | — | 1.13 | 0.50 |
| Total | 150.00 | 100.00 | 300.00 | 100.00 | 225.00 | 100.00 |
| Tablet Size/Shape | 9/32″ | Round | 0.486″ × 0.240″ | Oval | 5/16″ | Round |
| Tablet Thickness | 0.120″ | — | 0.200″ | — | 0.140″ | — |

| | 3 mg Formulation | | 3.5 mg Formulation | | 4 mg Formulation | |
|---|---|---|---|---|---|---|
| Component | mg/tablet | % w/w | mg/tablet | % w/w | mg/tablet | % w/w |
| Guanfacine Hydrochloride, | 3.42 | 1.71 | 3.99 | 1.71 | 4.56 | 1.71 |
| USP/NF | | | | | | |
| Hypromellose 2208, USP/NF | 20.00 | 10.00 | 23.33 | 10.00 | 26.67 | 10.00 |
| (Methocel ® K4M CR Premium) | | | | | | |
| High Density Silicified | 42.78 | 21.39 | 49.91 | 21.39 | 57.04 | 21.39 |
| Microcrystalline Cellulose | | | | | | |
| (Prosolv HD90 ™) | | | | | | |
| Lactose Monohydrate, | 42.80 | 21.40 | 49.93 | 21.40 | 57.07 | 21.40 |
| Povidone, Crospovidone | | | | | | |
| Granulated Blend (Ludipress ®) | | | | | | |
| Methacrylic Acid Copolymer, | 50.00 | 25.00 | 58.33 | 25.00 | 66.67 | 25.00 |
| Type C, USP/NF (Eudragit ® L | | | | | | |
| 100-55) | | | | | | |
| Fumaric Acid, FCC | 10.00 | 5.00 | 11.67 | 5.00 | 13.33 | 5.00 |
| Atomized Glyceryl Behenate, | 30.00 | 15.00 | 35.00 | 15.00 | 40.00 | 15.00 |
| USP/NF (Compritol ® 888 ATO) | | | | | | |
| Pigment Blend Powder* | 1.00 | 0.50 | 1.17 | 0.50 | 1.33 | 0.50 |
| Total | 200.00 | 100.00 | 233.33 | 100.00 | 266.66 | 100.00 |
| Tablet Size/Shape | 5/16″ | Round | 5/16″ | Round | 0.486″ × 0.240″ | Oval |
| Tablet Thickness | 0.130″ | — | 0.140″ | — | 0.170″ | — |

*2.5 mg: Blue pigment; 3 mg: Green pigment; 3.5 mg: Red pigment; 4 mg: Green pigment

Further aspects of the invention include formulations which combine two or more of the pharmacokinetic parameters, or sets of pharmacokinetic parameters (e.g., AUC₀₋ₗₐₛₜ, AUC_{∞}, Cₘₐₓ and/or Tₘₐₓ), described above, for example two, three, or four, etc., pharmacokinetic parameters. For example, such aspects include formulations which provide one or more of the pharmacokinetic parameters, or sets thereof, as shown herein when administered to adult patients and one or more of the pharmacokinetic parameters, or sets thereof, as shown herein when administered to children ages 6-12 and/or children ages 13-17. Preferred combinations of two or more pharmacokinetic parameters to be satisfied are AUC₀₋ₜ and Cₘₐₓ.

The formulations of this invention are useful over an extended period. By “extended period” is meant that the formulation of guanfacine of this invention can be administered to a patient for a length of time of about a week, a month, 2-11 months, (e.g., 2-6 months, 7-11 months, etc.), a year, two or more years, etc. and continue to be effective to treat the indication involved, e.g., ADHD.

In an advantageous embodiment, the pharmaceutical formulations of guanfacine comprise Eudragit L® 100-55 (poly(methacrylic acid, ethyl acrylate)), (anionic polymer of methacrylic acid and methacrylates—Methacrylic copolymer Type C, NF) marketed by Rohm America, Inc. All specification sheets available from Rohm America, Inc. by the filing date of this application for Eudragit L® 100-55 are hereby incorporated herein by reference. The compositions in this embodiment generally contain Eudragit L® 100-55, out of one hundred percent by weight of total composition (w/w), from about 25 percent to about 45 percent, preferably from about 25 percent to about 35 percent, most preferably from about 26, 27, 28, etc. percent to about 32, 33, 34, 35, etc. percent (w/w) of the total composition, the content optionally varying with dosage level as shown below.

A further aspect includes formulations which achieve the PK profile described above using formulations based on conventional galenical technology using osmotic release, pulsatile release, sustained release, controlled release, delayed release, extended release, immediate release or other modified release of guanfacine.

In other aspects, the invention is directed to methods for treatment using, as described above, guanfacine provided in a modified release formulation. The formulation is administered to an adult patient (18+ years), about 6-12 year olds, or about 13-17 year olds and achieves treatment of ADHD, hypertension, heroin withdrawal, certain problems in difficult pregnancies, sleep disorders, etc.

In yet another aspect, the invention pertains to a packaged pharmaceutical composition or formulation comprising a container holding a pharmaceutical composition comprising guanfacine and instructions for using the compound for an indication for guanfacine, e.g., ADHD, hypertension, heroin withdrawal, certain problems in difficult pregnancies, sleep disorders in a subject, etc.

The term “vehicle” has the broadest meaning, including any substance with which drugs are formulated.

The term “container” includes any receptacle for holding the therapeutic formulation. For example, in one embodiment, the container is the packaging that contains the formulation. In other embodiments, the container is not the packaging that contains the formulation, i.e., the container is a receptacle, such as a box or vial that contains the packaged formulation or unpackaged formulation and the instructions for use of the formulation. Moreover, packaging techniques are well known in the art. It should be understood that the instructions for use of the therapeutic formulation may be contained on the packaging containing the therapeutic formulation, and as such the instructions form an increased functional relationship to the packaged product. However, it should be understood that the instructions can contain information pertaining to guanfacine's ability to perform its intended function, e.g., treat ADHD, hypertension, heroin withdrawal, certain problems in difficult pregnancies, sleep disorders, etc.

Supplementary active compounds can also be incorporated into the compositions as long as they do not significantly affect the ability of the therapeutic formulation to perform its intended function or do not significantly affect the ability of the therapeutic formulation to achieve the intended plasma concentration pharmacokinetic (PK) profile as described above.

In certain embodiments of the invention, the subject is in need of treatment by the methods of the invention, and is selected for treatment based on this need. A subject in need of treatment is art-recognized, and includes subjects that have been identified as having a disease or disorder related to ADHD, hypertension, heroin withdrawal, certain problems in difficult pregnancies, sleep disorders, etc., having a symptom of such a disease or disorder, or at risk of such a disease or disorder, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder).

Administration of guanfacine to a subject to be treated can be carried out using known procedures, at dosages and for periods of time effective to achieve the intended treatment in the subject. An effective amount of the therapeutic compound necessary to achieve a therapeutic effect may vary according to factors such as the age, sex, and weight of the subject. Actual dosage levels of guanfacine in the pharmaceutical compositions of this invention may be varied so as to obtain an amount of the active ingredient that is effective to achieve the desired therapeutic response for a particular patient, composition, and mode of administration, without being toxic to the patient.

Depending on the formulation system to be used, pharmaceutically acceptable carriers include any of the following: fillers or extenders, such as sugars, starches, lactose, sucrose, glucose, mannitol, or silicic acid; binders, such as, for example, carboxymethylcellulose, alginates, gelatin, polyvinyl pyrrolidone, sucrose or acacia; humectants, such as glycerol; disintegrating agents, such as agar-agar, calcium carbonate, potato or tapioca starch, alginic acid, certain silicates, sodium starch glycolate, cross-linked sodium carboxymethyl cellulose, cross-linked sodium carboxymethyl cellulose (e.g., AC-DI-SOL®), sodium starch glycolate (e.g., EXPLOTAB®, PRIMOJEL®), and cross-linked polyvinylpolypyrrolidone (e.g., Plasone-XL), and sodium carbonate; solution retarding agents, such as paraffin; absorption accelerators, such as quaternary ammonium compounds; excipients, such as cocoa butter; solvents; encapsulating material; wetting agents, such as, for example, cetyl alcohol and glycerol monostearate; absorbents, such as kaolin and bentonite clay; lubricants such as talc, stearic acid, leucine, glyceryl behanate, hydrogenated vegetable oil, calcium stearate, magnesium stearate, solid polyethylene glycols, sodium lauryl sulfate; coloring agents; preservatives; surface-active agents; dispersing agent; inert liquid diluent; adjuvants; emulsifying agents; coating agents; sweetening, flavoring and/or perfuming agents; suppository waxes; oils, such as peanut oil, cottonseed oil, safflower oil, sesame oil, olive oil, corn oil and soybean oil; antioxidants, for example, water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (B HA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; sodium citrate; dicalcium phosphate; phosphate buffer solutions; and other non-toxic compatible substances employed in pharmaceutical formulations known in the art. Prevention of the action of microorganisms on the tablets may be ensured by the inclusion of various antibacterial and antifungal agents, for example, paraben, chlorobutanol, phenol sorbic acid, and the like. Each carrier must be “acceptable” in the sense of being compatible with the other ingredients of the formulation, not injurious to the patient, and in the sense that it does not affect the ability of the therapeutic formulation to achieve desired PK profiles.

As noted above, formulations for achieving the foregoing PK profiles dosing regimens can use immediate, controlled, sustained, extended, delayed, pulsatile, osmotic release, etc. technologies, alone or in combination to achieve the desired results. Examples of principles of formulations and preparations are contained, for example, in the Handbook of Pharmaceutical Excipents, American Pharmaceutical Association 4^{th} edition, 2003 (Rowe, Sheskey and Weller); Pharmaceutical Dosage Forms: Tablets (Lieberman, Lachman and Schwarts, editors) current edition, published by Marcel Dekker, Inc., as well as Remington's Pharmaceutical Sciences (A. Gennaro, editor, 20th edition, 2000). For conventional sustained or controlled release methods, see, e.g., in R. K. Chang and J. R. Robinson, chapter 4: “Sustained Drug Release from Tablets and Particles Through Coating,” in Pharmaceutical Dosage Forms: Tablets, volume 3, edited by H. A. Lieberman, L. Lachman, and J. B. Schwartz, Marcel Dekker, Inc., 1991; R. J. Campbell and G. L. Sackett, chapter 3: “Film coating,” in Pharmaceutical Unit Operations: Coating, edited by K. E. Avis, A. J. Shukla, and R. K. Chang, Interpharm Press, Inc., 1999. Principles of alternative formulation techniques are also given in U.S. Pat. Nos. 6,878,386; 6,849,661; 6,827,947; 6,702,803; 6,793,936; 6,780,436; 6,746,692; 6,638,535; 6,635,277; 6,627,223. All of these disclosures are entirely incorporated by reference herein.

Other guanfacine formulations different from those specifically exemplified herein can be provided in the form of beads, e.g., having a core which is optionally coated with a coating which allows the release of the agent immediately or over time, such as a pharmaceutically acceptable water-insoluble or water soluble film former alone or with a dissolution regulating agent etc. See, e.g., U.S. Pat. No. 4,728,512. A biphasic or multiphasic release profile can be achieved by combining the immediate-release beads with delayed, sustained or other controlled release beads or by providing various extended release beads with differing release profiles—all to achieve the desired PK profiles.

Beads can be prepared by coating conventional drug-containing cores with a water-insoluble polymer, or a combination of water-insoluble polymers, or a combination of water-insoluble and water-soluble polymers. This may be a combination of layers, or a combination of polymers in a single coating. The resultant beads (or tiny tablets) can then be placed in a capsule. Other than beads in a capsule shell, tablets in a capsule shell (e.g., one or more immediate-release tablet and one or more delayed, sustained release tablet in a capsule shell) also can be used to attain the desired release profile.

Various polymeric materials can be used to achieve the desired type of pattern of release, e.g., immediate, sustained, delayed etc. release. For example, a multiple dosage form (e.g., as discussed below) can deliver rapid and complete dosages of active agent to a recipient multiple times over a period of hours with a single oral administration, if it is desired to combine dosages in a single unit; additionally, doses with sustained or delayed release function can be combined with each other or with doses having immediate release functionality.

Examples of possible bead constructions are plentiful and include the following:
Sugar core bead, coated with active agent and then coated with polymer and/or with mix of active agent and polymer or any different order of such layers on the core, within each case, selected active agent concentrations of components in the layers.
Bead containing active agent core, coated with polymer, and/or with mix of active agent and polymer or any different order of such layers on the core, within each case, selected active agent concentrations of components in the layer.
Tablet or capsule containing multiple types of beads as described above having differing timing of release or different rates of release of active agent.

Matrix beads can also be used, i.e., not having any layers to achieve sustained or delayed release. The components used in such matrices are usually chosen from conventional sustained release or delayed release polymers.

Guanfacine-containing particles may also be incorporated into a tablet, in particular by incorporation into a tablet matrix, which rapidly disperses the particles after ingestion, in certain embodiments. In order to incorporate these particles into such a tablet, a filler/binder is added to a tablet that can accept the particles, but will not allow their destruction during the tableting process. Materials that are suitable for this purpose include, but are not limited to, microcrystalline cellulose (e.g., AVICEL®), soy polysaccharide (e.g., EMCOSOY®), pre-gelatinized starches (e.g., STARCH® 1500, NATIONAL® 1551), and polyethylene glycols (e.g., CARBOWAX®). The materials are typically present in the range of 5-75% (w/w), with a preferred range generally of 25-50% (w/w).

Various enteric materials, e.g., cellulose acetate phthalate, hydroxypropyl methylcellulose phthalate, polyvinyl acetate phthalate, and the EUDRAGIT® and ACRYLEZE® acrylic polymers, can also be employed. These are gastroresistant, enterosoluble coatings for drug release in the intestine when desired. The enteric materials, which are soluble at higher pH values, are frequently used for colon-specific delivery systems and are entirely conventionally employable in the systems of this invention. The enteric polymers used in this invention can also be modified conventionally by mixing with other known coating products that are not pH sensitive. Examples of such coating products include the neutral methacrylic acid esters with a small portion of trimethylammonioethyl methacrylate chloride, which are commercially available, e.g., EUDRAGIT® RS and EUDRAGIT® RL; neutral ester dispersions without any functional groups, e.g., EUDRAGIT® NE30D and EUDRAGIT® NE30; most preferably, EUDRAGIT® L100-55, and other pH independent coating products.

A conventional protective coating layer may also be applied immediately outside the core, either a drug-containing matrix core or a drug-layered core, by conventional coating techniques such as pan coating or fluid bed coating using solutions of polymers in water or suitable organic solvents or by using aqueous polymer dispersions. Suitable materials for the protective layer include cellulose derivatives such as hydroxyethyl cellulose, hydroxypropyl cellulose, hydroxypropyl methylcellulose, polyvinylpyrrolidone, polyvinylpyrrolidone/vinyl acetate copolymer, ethyl cellulose aqueous dispersions, polyvinyl acetate (e.g., AQUACOAT®, SURELEASE®), EUDRAGIT®'s, OPADRY® and the like. Typical coating levels are from 1 to 6%, in general, preferably 2-4% (w/w).

An overcoating layer can further optionally be applied to the compositions of the present invention. OPADRY®, OPADRY II® (Colorcon) and corresponding color and colorless grades from Colorcon can be used to protect the pellets from being tacky and provide colors to the product. Typical levels of protective or color coating are from 1 to 6%, in general preferably 2-3% (w/w). Many ingredients can be incorporated into the overcoating formula, for example to provide a quicker (immediate) release, such as plasticizers: acetyltriethyl citrate, triethyl citrate, acetyltributyl citrate, dibutylsebacate, triacetin, polyethylene glycols, propylene glycol and the others; lubricants: talc, colloidal silica dioxide, magnesium stearate, calcium stearate, titanium dioxide, magnesium silicate, and the like.

Optional modifying components of a protective layer which can be used over the enteric or other coatings include a water penetration barrier layer (semi-permeable polymer) which can be successively coated after the enteric or other coating to reduce the water penetration rate through the enteric coating layer and thus increase the lag time of the drug release. Sustained-release coatings commonly known to one skilled in the art can be used for this purpose by conventional coating techniques such as pan coating or fluid bed coating using solutions of polymers in water or suitable organic solvents or by using aqueous polymer dispersions. For example, the following materials can be used, but not limited to: cellulose acetate, cellulose acetate butyrate, cellulose acetate propionate, ethyl cellulose, fatty acids and their esters, waxes, zein, and aqueous polymer dispersions such as EUDRAGIT®'s, e.g., RS, RL 30D, NE 30D, AQUACOAT®, SURELEASE®, cellulose acetate latex, etc. Combinations of the above polymers and hydrophilic polymers such as hydroxyethyl cellulose, hydroxypropyl cellulose (KLUCEL®, Hercules Corp.), hydroxypropyl methylcellulose (METHOCEL®, Dow Chemical Corp.), and polyvinylpyrrolidone can also be used.

Details of using the foregoing constructs and others to achieve a desired release profile as discussed above are fully conventional and can be determined by those of skill in the art with at most a few routine parametric experiments, and conventional adjustments, e.g., involving identities of polymers and mixtures thereof, relative amounts of components, coating thicknesses, bead diameters, number of layers and compositions thereof, etc. Thus, for example, for a given construct, in vitro dissolution profiles can be determined. Fully conventional formulation and dissolution profile adjustments can be made routinely. Formulations having the desired in vitro release profiles produce the desired plasma concentration levels. These plasma profiles will be correlated with the in vitro release profiles in view of the usual factors, e.g., in vivo dissolution and absorption properties, active agent half-lives, etc., which correlation is well understood in the art.

Suitable materials which can be used to achieve formulations having such release profiles are well known and include but are not limited to polyvinyl acetate, cellulose acetate, cellulose acetate lattices, cellulose acetate butyrate, cellulose acetate propionate, ethyl cellulose, fatty acids and their esters, alkyl alcohols, waxes, zein (prolamine from corn), and aqueous polymeric dispersions such as the commercially available Eudragit®, Aquacoat®, Surelease®, Kollicoat®, etc., products.

The tablets (and other possible solid dosage forms of the pharmaceutical compositions of the present invention, such as dragees, capsules, pills, pellets, and granules,) may optionally be scored.

In general, the pharmaceutical formulations comprise 0.1 to 40% by weight of any single ingredient, more typically 0.5-35% by weight, depending on the formulation technology employed.

Definition of Terms

Cₘₐₓ—the maximum observed plasma concentration.
Tₘₐₓ—the time of occurrence of Cₘₐₓ.
AUC₀₋ₗₐₛₜ—the area under the plasma concentration versus time curve from time zero to the last sampling time at which concentrations were at or above the limit of quantification, calculated by the linear trapezoidal rule.
AUC_{∞}—the area under the plasma concentrations versus time curve from time zero to infinity, calculated from AUC₀₋ₜ+(Cₗₐₛₜ/λ_{Z}), where Cₗₐₛₜ is the last observed quantifiable concentration and λz is the apparent terminal rate constant.
t_{1/2}—the apparent terminal half-life, calculated from ln2/λ_{Z}.
API—Active pharmaceutical ingredient
Fasted—Overnight fasting, e.g., usually about 10-12 hours.

### BRIEF DESCRIPTION OF THE DRAWINGS

FIG. 1 shows the range of hardness at different compression forces for formulations with different Eudragit polymers.

The contents of all references, issued patents, and published patent applications cited throughout this application are hereby incorporated by reference. It should be understood that the use of any of the compounds described herein or in the applications identified in “The Related Applications” Section are within the scope of the present invention and are intended to be encompassed by the present invention and are expressly incorporated herein at least for these purposes, and are furthermore expressly incorporated for all other purposes.

Those skilled in the art will recognize, or be able to ascertain using no more than routine experimentation and the content of the instant specification, numerous equivalents to the specific procedures, embodiments, claims, and examples described herein. Such equivalents are considered to be within the scope of this invention and covered by the claims appended hereto.

It is to be understood that wherever values and ranges are provided herein, e.g., in ages of subject populations, dosages, and blood levels, all values and ranges encompassed by these values and ranges, are meant to be encompassed within the scope of the present invention. Moreover, all values that fall within these ranges, as well as the upper or lower limits of a range of values are also contemplated by the present application.

### EXAMPLES

The invention is further illustrated by the following examples which should not be construed as further limiting the subject invention.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated. The particle size range of the guanfacine HCl (as the API) used in the foregoing examples, tables and figures, is dv50 of 5 microns to 25 microns and dv90 of 10 microns to 50 microns. The term “dv” refers to volume diameter (the diameter of a sphere having the same volume) and 50 means 50 percent of the particles and 90 means 90 percent of the particles

### Example 1

The primary pieces of equipment used for manufacturing the tablets are a 16 quart V-shaped blender equipped with an intensifier bar and a 16 station rotary tablet press. All materials are passed through a 40 mesh screen and charged into a 16 quart V-blender, with guanfacine sandwiched in the middle. The mix is blended for ten minutes, with the intensifier bar turned on for minutes 5-8. The blend is charged into a polyethylene bag and then transferred to the hopper of the Stokes tablet press. The blend is compressed to the appropriate hardness for the necessary tablet weight. Tablet hardness is tested with a Schleuniger hardness tester.

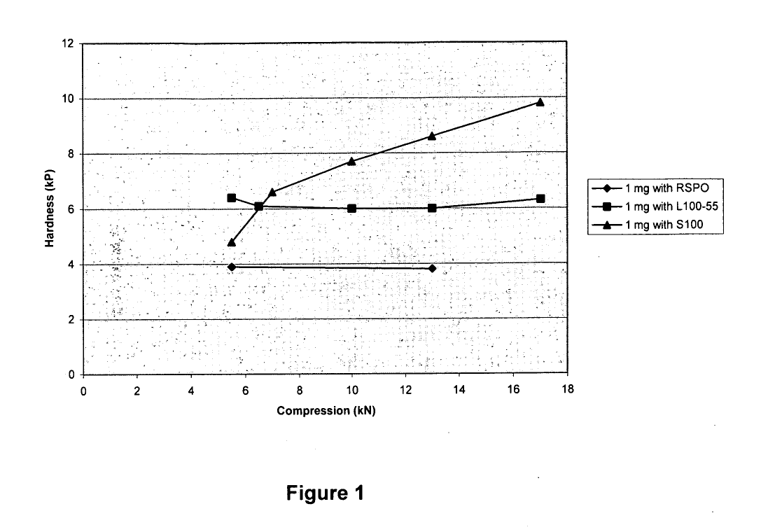

Formulations containing either Eudragit L100-55 or Eudragit RSPO were tested for hardness at various compression forces (see FIG. 1).

| | Composition 1 | Composition 2 |
|---|---|---|
| Ingredients | (% w/w) | (% w/w) |
| Guanfacine HCl | 0.76 | 0.76 |
| Methocel K4M* | 13.34 | 13.34 |
| Eudragit L100-55* | 33.33 | N/A |
| Eudragit RSPO | N/A | 33.33 |
| Fumaric acid | 5.00 | 5.00 |
| Compritol 888 ATO* | 13.33 | 13.33 |
| Ludipress* | 16.91 | 16.91 |
| Prosolv HD90* | 17.33 | 17.33 |

*Methocel K4M—Hydroxypropylmethylcellulose, Dow Chemical; Eudragit L100-55-poly(methacrylic acid, ethyl acrylate), a methacrylic acid copolymer, Rohm America, Inc.; Eudragit RSPO—Ammonio Methacrylate copolymer, Rohm America, Inc.; Compritol 888 ATO—Glyceryl dibehenate, Gattefosse; Ludipress—Lactose/povidone/crospovidone, BASF; Prosolv HD90—Silicified microcrystalline cellulose, Penwest.

| Composition | Average Hardness (kP) |
|---|---|
| 1 (Eudragit L100-55) | 6.2 |
| 2 (Eudragit RSPO) | 3.9 |

The general method for preparing guanfacine tablets used herein is similar to the method for the same in U.S. Pat. No. 6,811,794 and U.S. Pat. No. 6,287,599.

### Example 2

Objective: The objective of this study was to assess the pharmacokinetics of controlled-release guanfacine after a single 2 mg dose or multiple doses of 2 mg and 4 mg from Table 2.

Methods: An open-label, dose-escalation study in male and female children (aged 6-12 years) and adolescents (aged 13-17 years) with ADHD. Subjects weighed 25 kg (55 lb) or more but were not considered overweight in the opinion of the investigator. Their electrocardiogram readings (ECGs) were within normal ranges, and their blood pressure measurements were within the 95th percentile for their age, height, and gender. Patients were excluded if they had a comorbid psychiatric diagnosis, a history or family history of cardiac disease that the investigator judged to put the patient at risk, or were taking a CYP3A4- or P450-affecting agent. Informed consent was given by the patient's parent or guardian.

Patients received a single 2 mg dose on day 1 followed by repeated 2 mg qd (once daily) doses on Days 9-15, repeated 3 mg qd doses on Days 16-23, and repeated 4 mg qd doses on Days 24-29. The dose was then titrated down to 3 mg on days 30 to 32, and 2 mg on days 33 to 35. Pharmacokinetic assessments were made after the single 2 mg dose and after 6 days of repeated 2 mg and 4 mg doses.

Results: All 28 enrolled patients completed the trial. Demographic data are provided in the following table.

Demographic information

| | Children | Adolescents | Total |
|---|---|---|---|
| | (n = 14) | (n = 14) | (N = 28) |
| Age, mean (SD), y | 9.3 ± 1.82 | 14.2 ± 1.05 | 11.8 ± 2.90 |
| Gender | | | |
| Male, % | 50 | 85.7 | 67.9 |
| Female, % | 50 | 14.3 | 32.1 |
| Race | | | |
| White, % | 64.3 | 85.7 | 75 |
| African-American % | 28.6 | 7.1 | 17.9 |
| Other, % | 7.1 | 7.1 | 7.1 |
| Weight, mean (SD) | 76.6 ± 23.78 | 125.9 ± 20.87 | 101.2 ± 33.34 |
| Height, mean (SD) | 54.3 ± 4.59 | 65.1 ± 3.93 | 59.7 ± 6.91 |
| Body mass index, | 17.8 ± 2.60 | 20.8 (2.33) | 19.3 ± 2.85 |
| mean (SD), kg/m² | | | |

Pharmacokinetic findings are summarized in the following tables.

Summary of pharmacokinetic parameters for guanfacine after oral administration of 2 mg on Day 1 to children (6-12 years) and adolescent (13-17 years) ADHD patients

| | Children | Adolescents |
|---|---|---|
| | (6-12 yrs) | (13-17 yrs) |
| Parameter* | (N = 14) | (N = 14) |
| Cₘₐₓ (ng/mL) | 2.55 ± 1.03 | 1.69 ± 0.43 |
| Tₘₐₓ (h) | 4.98 | 4.96 |
| AUC₀₋ₜ (h · ng/mL) | 56.9 ± 22.0 | 42.7 ± 12.9 |
| AUC_{0-∞}(h · ng/mL) | 65.2 ± 23.9 | 47.3 ± 13.7 |
| λ_{z} (h⁻¹) | 0.0496 ± 0.0093 | 0.0428 ± 0.0153 |
| T_{1/2} (h) | 14.4 ± 2.39 | 17.9 ± 5.77 |
| CL/F (mL/min) | 612 ± 252 | 815 ± 214 |
| V_{z}/F (L) | 766 ± 380 | 1,239 ± 432 |

Summary of pharmacokinetic parameters for guanfacine on Day 14 after oral administration of 2 mg once daily to children (6-12 years) and adolescent (13-17 years) ADHD patients.

| | Children | Adolescents |
|---|---|---|
| | (6-12 yrs) | (13-17 yrs) |
| Parameter* | (N = 14) | (N = 14) |
| Cₘₐₓ (ng/mL) | 4.39 ± 1.66 | 2.86 ± 0.77 |
| Tₘₐₓ (h) | 4.98 | 4.53 |
| AUC₀₋₂₄ (h · ng/mL) | 70.0 ± 28.3 | 48.2 ± 16.1 |
| CL/F (mL/min) | 552 ± 215 | 826 ± 486 |

Summary of pharmacokinetic parameters for guanfacine on Day 28 after oral administration of 4 mg once daily to children (6-12 years) and adolescent (13-17 years) ADHD patients.

| | Children | Adolescents |
|---|---|---|
| | (6-12 yrs) | (13-17 yrs) |
| Parameter* | (N = 14) | (N = 14) |
| Cₘₐₓ (ng/mL) | 10.1 ± 7.09 | 7.01 ± 1.53 |
| Tₘₐₓ (h) | 5.02 | 4.97 |
| AUC₀₋₂₄ (h · ng/mL) | 162 ± 116 | 117 ± 28.4 |
| CL/F (mL/min) | 522 ± 212 | 607 ± 166 |

There were no discontinuations due to adverse events (AEs). No clinically significant abnormalities were observed on ECGs, hematologic parameters, or serum chemistries. Most AEs were mild to moderate in severity. The only AE to occur in ≧10% of subjects and considered possibly or probably due to treatment was somnolence (93%); only 6 subjects (21%) were judged to have severe somnolence.

Conclusions: The pharmacokinetics of guanfacine are linear after oral administration of single 2 mg doses and multiple 2 mg and 4 mg doses in both children (6-12 years) and adolescent (13-17 years) ADHD patients. Plasma concentrations and concentration-related pharmacokinetic parameters in children (6-12 years) are higher than those in adolescents (13-17 years) and, regardless of age group, are higher in female patients than in male patients. This is most likely due to the higher body weight in the adolescents compared to children and in males compared to females, regardless of age group. AEs were generally mild to moderate in severity and did not lead to any discontinuations. Controlled-release guanfacine appears to be a safe option for the treatment of ADHD in children and adolescents.

### Example 3

Objectives: The study was designed to assess the bioequivalence of guanfacine 2 mg and 4 mg tablets from table 2 and to investigate the dose proportionality of 1 mg, 2 mg, and 4 mg doses.

Methods: This was a Phase I, randomized, open-label, single-dose, five period, four-treatment crossover design with a separate lead-in period.

Forty-nine (49) healthy adult volunteers completed the study. During period 1, all subjects received a single 1 mg dose administered after and overnight fast. Prior to period 2, subjects were randomly assigned to one of four sequence groups, with 13 subjects per sequence. During treatment periods 2-5, subjects received their assigned treatment of 2 mg or 4 mg. All subjects were to receive two single doses of 2 mg tablets and two single doses of 4 mg tablets during the course of the study. Doses during periods 2-5 were also administered after an overnight fast.

Demographics: Gender:
Males: 28; Females: 24
Race: White: 7; Black: 5; Hispanic: 40; Other: 0
Overall mean age in years: 33. Age range 18-55 inclusive

Results: A summary of the pharmacokinetic data is shown below:

Summary of PK parameters for guanfacine after single oral administration of 1 mg, 2 mg and 4 mg tablets.

| | 1 mg | 2 mg (#1) | 2 mg (#2) | 4 mg (#1) | 4 mg (#2) |
|---|---|---|---|---|---|
| Parameter*† | (N = 52) | (N = 50) | (N = 50) | (N = 49) | (N = 49) |
| Cₘₐₓ (ng/mL) | 0.98 ± 0.26 | 1.59 ± 0.49 | 1.54 ± 0.53 | 3.58 ± 1.39 | 3.59 ± 1.40 |
| tₘₐₓ (h) | 6.00 | 6.00 | 6.00 | 5.01 | 6.00 |
| AUC₀₋ₜ (h · ng/mL) | 29.3 ± 8.84 | 55.0 ± 18.0 | 54.0 ± 17.6 | 120 ± 41.5 | 119 ± 43.4 |
| AUC_{0-∞} (h · ng/mL) | 32.4 ± 8.78 | 58.1 ± 18.8 | 57.8 ± 19.1 | 125 ± 46.0 | 123 ± 44.6 |
| λ_{z} (h⁻¹) | 0.0416 ± 0.0088 | 0.0439 ± 0.0075 | 0.0434 ± 0.0086 | 0.0434 ± 0.0098 | 0.0448 ± 0.0089 |
| t_{1/2} (h) | 17.5 ± 3.83 | 16.4 ± 3.46 | 16.7 ± 4.12 | 17.1 ± 5.51 | 16.2 ± 4.15 |
| CL/F (mL/mm) | 560 ± 194 | 647 ± 253 | 638 ± 206 | 599 ± 213 | 638 ± 331 |
| V_{z}/F (L) | 823 ± 249 | 894 ± 328 | 889 ± 241 | 854 ± 294 | 866 ± 393 |

Conclusions: The pharmacokinetics of guanfacine were reasonably linear over doses of 1 mg, 2 mg, and 4 mg although mean values for Cₘₐₓ and AUC₀₋ₗₐₛₜ and AUC_{inf} increased somewhat less than two fold between the 1 mg and 2 mg doses.

The 2 mg and 4 mg guanfacine tablets from two different data sets, i.e., (#1) and (#2), were bioequivalent to their respective strength tablets.

There were no deaths or serious adverse events.

### Example 4

Objectives: The objective of this study was to investigate the bioequivalence of guanfacine 2.5 mg tablets versus guanfacine 2 mg tablets.

Methods: This Phase I study utilized a randomized, open-label, single-dose, three-treatment crossover design. Forty-eight (48) healthy adult volunteers completed the study. All subjects were randomly assigned to one of 6 treatment sequences and received a 2 mg and 2.5 mg tablet of guanfacine. During treatment periods 1-3, subjects received their assigned treatment (a single oral dose 2 mg or 2.5 mg after an overnight fast).

Demographics: Gender:
Males: 14; Females: 34
Race: White: 10; Black: 1; Hispanic: 36; Other: 1
Overall mean age in years: 33

Results: A summary of the pharmacokinetic data is shown below:

Summary of PK parameters for guanfacine after oral administration of single 2 mg and 2.5 mg doses to healthy adult subjects.

| Parameter | 2 mg | 2.5 mg (#1) | 2.5 mg (#2) |
|---|---|---|---|
| Cₘₐₓ (ng/mL) | 1.71 ± 0.56 | 2.26 ± 0.67 | 2.49 ± 0.93 |
| Tₘₐₓ (hr) | 6 | 5 | 6 |
| AUC₀₋ₗₐₛₜ (ng · hr/mL) | 59.8 ± 20.9 | 77.8 ± 30.4 | 81.3 ± 35.4 |
| AUC_{0-inf} (ng · hr/mL) | 64.2 ± 22.6 | 84.5 ± 34.2 | 85.0 ± 37.4 |
| λz (h⁻¹) | 0.043 ± 0.012 | 0.040 ± 0.012 | 0.046 ± 0.011 |
| T½ (hr) | 17.7 ± 5.8 | 19.0 ± 6.6 | 16.7 ± 7.4 |
| CL/F (mL/min) | 612 ± 303 | 598 ± 299 | 591 ± 283 |
| Vz/F (L) | 884 ± 382 | 915 ± 390 | 809 ± 406 |

Arithmetic mean±standard deviation except for Tₘₐₓ for which the median is reported.

There were no deaths.

Conclusions: When corrected for the difference in dose, the 2.5 mg guanfacine tablets from the two data sets, i.e., (# 1) and (#2), are each bioequivalent to the 2 mg guanfacine tablets. The tablets appeared to be safe and generally well tolerated by the healthy male and female subjects in this study when administered in single oral 2 mg and 2.5 mg doses.

### Example 5

Osmotically Controlled Guanfacine Extended Release Tablets

The primary pieces of equipment useful for manufacturing osmotic tablets are a 16 quart V-shaped blender equipped with an intensifier bar, pan coater capable of solvent coating, a 16 station rotary tablet press, and laser hole drilling system to create the orifice from which drug is released. All materials including osmagents (Xylitol, Maltrin, and Mannitol) are passed through a 20 mesh screen and charged into a 16 quart V-blender, with guanfacine sandwiched in the middle. The blend is charged into a polyethylene bag and then transferred to the hopper of a Stokes tablet press. The blend is compressed to the appropriate hardness for the necessary tablet weight. Tablet hardness is tested with a Schleuniger hardness tester. Tablets are coated in a pan coater with spray rate of 60-100 g/min or higher. The coating solution is prepared by dissolving about 5% cellulose Acetate, NF (National Formulary) in Acetone then adding 25-45% plasticizers such as TEC. A laser drilled hole is then placed on one side of the tablets to allow for drug release. The tablet achieves the desired PK profile.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

We claim:

1. A pharmaceutical formulation comprising an effective amount of guanfacine or a pharmaceutically acceptable salt thereof and at least one vehicle, satisfactory to achieve effective once a day adminstration of said guanfacine to
a human subject who is at least 18 years old and satisfactory to achieve for said guanfacine for a single dose administration under fasting conditions, for a dose of 1 mg of guanfacine an AUC₀₋ₗₐₛₜ of about 29.3±20% ng·h/mL, a Cₘₐₓ of about 0.98±20% ng/mL, or for doses of 2 mg or another dose amount whose PK profile is linearly correlated with that of a 1 mg dose, an AUC₀₋ₗₐₛₜ and Cₘₐₓ linearly related to those for the 1 mg dose, wherein the total formulation weight for a 1 mg dose is up to 170 mg and for a 2 mg dose or other dose linearly correlated with a 1 mg dose, a total tablet weight linearly proportional thereto,
or
a human subject who is at least 18 years old and satisfactory to achieve for said guanfacine for a single dose administration under fasting conditions, for a dose of 2.5 mg of guanfacine an AUC₀₋ₗₐₛₜ of about 81.3±20% ng·h/mL, a Cₘₐₓ of about 2.49±20% ng/mL, or for another dose amount whose PK profile is linearly correlated with that of a 2.5 mg dose, an AUC₀₋ₗₐₛₜ and Cₘₐₓ linearly related to those for the 2.5 mg dose, wherein the total formulation weight for a 2.5 mg dose is up to 255 mg and for other dose linearly correlated with a 2.5 mg dose, a total tablet weight linearly proportional thereto,
or
a human subject who is at least 18 years old and satisfactory to achieve for said guanfacine for a single dose administration under fasting conditions, for a dose of 4 mg of guanfacine an AUC₀₋ₗₐₛₜ of about 120±20% ng·h/mL a Cₘₐₓ of about 3.58±20% ng/mL, or for doses of 3 mg, 3.5 mg or another dose amount whose PK profile is linearly correlated with that of a 4 mg dose, an AUC₀₋ₗₐₛₜ and Cₘₐₓ linearly related to those for the 4 mg dose, wherein the total formulation weight for a 4 mg dose is up to 300 mg and for a 3 mg dose, 3.5 mg dose or other dose linearly correlated with a 4 mg dose, a total tablet weight linearly proportional thereto,
or
a human patient who is 6-12 years old and satisfactory to achieve for said guanfacine for a single dose administration under fasting conditions, for a dose of 2 mg of guanfacine an AUC₀₋ₗₐₛₜ of about 56.88±20% ng·h/mL, a Cₘₐₓ of about 2.55±20% ng/mL, or for doses of 1 mg or another dose amount whose PK profile is linearly correlated with that of a 2 mg dose, an AUC₀₋ₗₐₛₜ and Cₘₐₓ linearly related to those for the 2 mg dose, wherein the total formulation weight for a 2 mg dose is up to 340 mg and for a 1 mg dose or other dose linearly correlated with a 2 mg dose, a total tablet weight linearly proportional thereto,
or
a human patient who is 13-17 years old and satisfactory to achieve for said guanfacine for a single dose administration under fasting conditions, for a dose of 2 mg of guanfacine an AUC₀₋ₗₐₛₜ of about 42.74±20% ng·h/mL, a Cₘₐₓ of about 1.69±20% ng/mL, or for doses of 1 mg or another dose amount whose PK profile is linearly correlated with that of a 2 mg dose, an AUC₀₋ₗₐₛₜ, and Cₘₐₓ linearly related to those for the 2 mg dose, wherein the total formulation weight for a 2 mg dose is up to 340 mg and for a 1 mg dose or other dose linearly correlated with a 2 mg dose, a total tablet weight linearly proportional thereto.

2. A pharmaceutical formulation of claim 1, which is in the form of a tablet.

3. A pharmaceutical formulation of claim 2, which is in the form of a tablet, wherein when the tablet is round it has a diameter of up to 0.4 inch with a thickness of up to 0.2 inch and when the tablet is oval it has a length of up to 0.6 inch and a width of up to 0.2 inch with a thickness of up to 0.25 inch.

4. A pharmaceutical formulation of claim 2, which is in the form of a tablet, wherein when the tablet is round it has a diameter of 0.1500 inch to 0.3125 inch with a thickness of 0.01000 to 0.01400 inch and when the tablet is oval it has a length of 0.4860 to 0.5400 inch and a width of 0.1200 to 0.2400 inch with a thickness of 0.0140 to 0.020 inch.

5. A pharmaceutical formulation of claim 1, wherein the human subject who is at least 18 years old is healthy not suffering from ADHD, and the human patient who is 6-12 and 13-17 years old is suffering from ADHD.

6. A pharmaceutical formulation of claim 2, which is in the form of a tablet, comprising an effective amount of guanfacine or a pharmaceutically acceptable salt thereof and at least one vehicle, satisfactory to achieve effective once a day administration of said guanfacine to a human patient, wherein the tablet size
for a 1 mg dose round tablet the diameter is up to about 9/23 inch,
for 2 mg and 4 mg dose oval tablets the lengths are up to about 0.486 inch and the widths are up to about 0.240 inch, and
for 2.5 mg, 3 mg and 3.5 mg dose round tablets the diameters are up to about 5/16 inch.

7. A pharmaceutical formulation of claim 6 wherein the respective tablet weight for a
1 mg dose is up to about 150 mg,
2 mg dose is up to about 300 mg,
2.5 mg dose is up to about 225 mg,
3 mg dose is up to about 200 mg,
3.5 mg dose is up to about 233.3 mg, and
4 mg dose is up to about 266.7 mg.

8. A pharmaceutical formulation of claim 1 comprising an anionic polymer of methacrylic acid and methacrylates—methacrylic copolymer Type C, NF.

9. A pharmaceutical formulation of claim 7 wherein the amount of the anionic polymer of methacrylic acid and methacrylates—methacrylic copolymer Type C, NF is about 25 to about 45% w/w.

10. A pharmaceutical formulation according to claim 1, wherein the formulation is effective to treat ADHD, hypertension, heroin withdrawal, certain problems in difficult pregnancies, and/or sleep disorders.

11. A method of treating ADHD, hypertension, heroin withdrawal, certain problems in difficult pregnancies, and/or sleep disorders comprising administering to a patient in need thereof a pharmaceutical composition according to claim 1.

12. A method according to claim 11, wherein ADHD is treated.

13. A method according to claim 11, wherein the patient is 18+years old.

14. A method according to claim 11, wherein the patient is 6-12 years old.

15. A method according to claim 11, wherein the patient is 13-17 years old.

16. A method according to claim 11, wherein administration is for an extended period.

17. A pharmaceutical formulation comprising an effective amount of guanfacine or a pharmaceutically acceptable salt thereof and at least one vehicle, satisfactory to achieve effective once a day adminstration of said guanfacine to
a human subject who is at least 18 years old and satisfactory to achieve for said guanfacine for a single dose administration, for a dose of 1 mg of guanfacine an AUC₀₋ₗₐₛₜ of about 29.3±20% ng·h/mL, a Cₘₐₓ of about 0.98±20% ng/mL, or for doses of 2 mg or another dose amount whose PK profile is linearly correlated with that of a 1 mg dose, an AUC₀₋ₗₐₛₜ and Cₘₐₓ linearly related to those for the 1 mg dose, wherein the total formulation weight for a 1 mg dose is up to 170 mg and for a 2 mg dose or other dose linearly correlated with a 1 mg dose, a total tablet weight linearly proportional thereto,
or
a human subject who is at least 18 years old and satisfactory to achieve for said guanfacine for a single dose administration, for a dose of 2.5 mg of guanfacine an AUC₀₋ₗₐₛₜ of about 81.3±20% ng·h/mL, a Cₘₐₓ of about 2.49±20% ng/mL, or for another dose amount whose PK profile is linearly correlated with that of a 2.5 mg dose, an AUC₀₋ₗₐₛₜ and Cₘₐₓ linearly related to those for the 2.5 mg dose, wherein the total formulation weight for a 2.5 mg dose is up to 255 mg and for other dose linearly correlated with a 2.5 mg dose, a total tablet weight linearly proportional thereto,
or
a human subject who is at least 18 years old and satisfactory to achieve for said guanfacine for a single dose administration, for a dose of 4 mg of guanfacine an AUC₀₋ₗₐₛₜ of about 120±20% ng·h/mL a Cₘₐₓ of about 3.58±20% ng/mL, or for doses of 3 mg, 3.5 mg or another dose amount whose PK profile is linearly correlated with that of a 4 mg dose, an AUC₀₋ₗₐₛₜ and Cₘₐₓ linearly related to those for the 4 mg dose, wherein the total formulation weight for a 4 mg dose is up to 300 mg and for a 3 mg dose, 3.5 mg dose or other dose linearly correlated with a 4 mg dose, a total tablet weight linearly proportional thereto,
or
a human patient who is 6-12 years old and satisfactory to achieve for said guanfacine for a single dose administration, for a dose of 2 mg of guanfacine an AUC₀₋ₗₐₛₜ of about 56.88±20% ng·h/mL, a Cₘₐₓ of about 2.55±20% ng/mL, or for doses of 1 mg or another dose amount whose PK profile is linearly correlated with that of a 2 mg dose, an AUC₀₋ₗₐₛₜ and Cₘₐₓ linearly related to those for the 2 mg dose, wherein the total formulation weight for a 2 mg dose is up to 340 mg and for a 1 mg dose or other dose linearly correlated with a 2 mg dose, a total tablet weight linearly proportional thereto,
or
a human patient who is 13-17 years old and satisfactory to achieve for said guanfacine for a single dose administration, for a dose of 2 mg of guanfacine an AUC₀₋ₗₐₛₜ of about 42.74±20% ng·h/mL, a Cₘₐₓ of about 1.69±20% ng/mL, or for doses of 1 mg or another dose amount whose PK profile is linearly correlated with that of a 2 mg dose, an AUC₀₋ₗₐₛₜ, and Cₘₐₓ linearly related to those for the 2 mg dose, wherein the total formulation weight for a 2 mg dose is up to 340 mg and for a 1 mg dose or other dose linearly correlated with a 2 mg dose, a total tablet weight linearly proportional thereto.
